(19) United States
(12) Patent Application Publication
(10) Pub. No.: US 2021/0094966 A1
(43) Pub. Date: Apr. 1, 2021

(54) PROCESS FOR PRODUCING A FIVE-MEMBERED POLYCYCLOALIPHATIC CARBONATE

(71) Applicants: TEREOS STARCH & SWEETENERS BELGIUM, Aalst (BE); CENTRE NATIONAL DE LA RECHERCHE SCIENTIFIQUE, Paris Cedex 16 (FR); SOPREMA, Strasbourg Cedex (FR); UNIVERSITÉ DE STRASBOURG, Strasbourg Cedex (FR)

(72) Inventors: Pierre-Étienne BINDSCHEDLER, Strasbourg Cedex (FR); Rémi PERRIN, Strasbourg Cedex (FR); Pierre FURTWENGLER, Paris Cedex 16 (FR); Luc AVÉROUS, Paris Cedex 16 (FR); Andreas REDL, Aalst (BE)

(73) Assignees: TEREOS STARCH & SWEETENERS BELGIUM, Aalst (BE); CENTRE NATIONAL DE LA RECHERCHE SCIENTIFIQUE, Paris Cedex 16 (FR); SOPREMA, Strasbourg Cedex (FR); UNIVERSITÉ DE STRASBOURG, Strasbourg Cedex (FR)

(21) Appl. No.: 16/499,441

(22) PCT Filed: Mar. 30, 2018
(86) PCT No.: PCT/EP2018/058343

§ 371 Date: Sep. 30, 2019

(30) Foreign Application Priority Data
Mar. 31, 2017 (FR) 1700351

(51) Int. Cl. C07D 493/04; C08G 71/04; C08G 65/26; C08G 64/02

(52) U.S. Cl. CPC C07D 493/04; C08G 71/04; C08G 64/0208; C08G 65/2606; C08G 65/2621

(57) ABSTRACT

A process for producing a five-membered polycycloaliphatic carbonate including a step of reacting a suspension of a powdered sugar alcohol within a carbon dioxide source and a catalyst compound which is soluble in the carbon dioxide source at the reactional temperature. The invention also concerns a process for producing non-isocyanate polyurethane and/or a polycarbonate and/or polyethers including the step of obtaining a five-membered polycycloaliphatic carbonate according to the process for producing a five-membered polycycloaliphatic carbonate.

### FIELD OF INVENTION

The present invention concerns a solvent free process for producing five-membered polycycloaliphatic carbonate from D-Sorbitol.

### BACKGROUND OF INVENTION

D-sorbitol is a sugar alcohol which can be obtained from D-glucose hydrogenolysis. D-sorbitol is a remarkable building block quoted as one of the top twelve renewable chemicals of added-value from biomass^{1,2} offering lots of opportunities due to its high functionality. To ensure new ecological concern like global warming, wastes management and side product valorization a fruitful alliance between chosen building blocks working as reactant and solvent at lower temperature, with a simple reaction work-up is highly requested in academic and industrial field.

D-sorbitol can be directly used has monomers in polymer synthesis through classical esterification pathway³ or more recently via enzymatic synthesis as reported by Liliana Gustini and colleagues for coating applications. Nevertheless, to extend the development of advanced oligomers or polymers, it is highly demanded to develop new D-sorbitol based molecules like five-membered bis(cyclo-carbonate) (BisCC) to synthetized poly(hydroxyurethane) (PHUs) also called non-isocyanate polyurethane (NIPU)⁴⁻⁷, polyol polyether⁸ or polycarbonate.

Aliphatic polycarbonates were synthesized at low temperature (≤60° C.) from five-membered BisCC via anionic ring opening polymerization (ROP) in particular case^{9,10} but most of five-membered BisCC are thermodynamically unfavorable and generally proceeds at high temperature (≈150° C.) leading to the elimination of carbon dioxide to produce linear copolymers with carbonates and ethers linkage.

A wise reactant to be associated with D-sorbitol is dimethyl carbonate (DMC) which can be an environmentally friendly reactive solvent according to is nontoxicity and represents an alternative to phosgene for methylation and carbonylation processes¹¹. DMC with 99.8% purity is currently produced through the Enichem^{12,13} process and represents 85% of Europe's production¹⁴. The Enichem process implies carbon monoxide and oxygen as building blocks for the production of DMC. The future will be even better as promising new production ways based on CO₂ widely available in the environment, positioning DMC as rising green reactive solvent. The association of DMC and D-sorbitol has already been reported by Karolina M. Tomczyk working with 10 eq. of DMC toward D-sorbitol using 1.4-dioxane as solvent and potassium carbonate as catalyst at 80° C. The obtained product was (1R,4S,5R,6R)-6-(1,3-dioxolan-2-one-4-yl)-2,4,7trioxa-3-oxy-bicyclo [3.3.0]octane, a BisCC with a yield of 43% after recrystallization in acetonitrile. More recently Magdalena M. Mazurek-Budzynska also obtained this molecule working with 10 eq. of DMC toward D-sorbitol in methanol catalyzed by potassium carbonate for a global yield of 40%. Otherwise in the past, other sugar alcohols have been transformed in five members cyclic(carbonate) under different conditions like D-glucitol tricarbonate synthesized from diphenyl carbonate in N,N-dimethylformamide and catalyzed by sodium hydrogen carbonate at 110° C.¹⁵ and D-mannitol tricarbonate was synthesized under harsh conditions from D-mannitol and ethylene glycol at 150° C./20 mmHg¹⁶.

However, five-membered polycycloaliphatic carbonates were always obtained from sugar alcohols and linear or cyclic carbonates using solvents and/or harsh conditions. Solvents such as 1.4-dioxane are potentially explosive, skin/eyes irritating and toxic (affecting central nervous system, liver, kidneys and anticipated to be a human carcinogen).

Consequently, there is a huge demand to develop a solvent-free process for obtaining five-membered polycycloaliphatic carbonates from biosourced substrates such as sugar alcohols.

### SUMMARY

The inventors have developed and optimized a solvent-free process providing the synthesis of a five-membered polycycloaliphatic carbonate by using a powdered sugar alcohol. The inventors also developed a process using as carbon dioxide source a green and harmless reactive compound that can be used in a solid—liquid reaction, a process providing a five-membered polycycloaliphatic carbonate in a relatively pure form and valuable subsidiary products such as methanol, ethanol, ethylene glycol or 2,3 propane diol.

The invention concerns a process for producing a five-membered polycycloaliphatic carbonate comprising a step of reacting a suspension of a powdered sugar alcohol, typically a crystalline sugar alcohol within a carbon dioxide source and a catalyst compound which is soluble in said carbon dioxide source at the reactional temperature.

In one embodiment, the powdered sugar alcohol selected among erythritol, arabitol, xylitol, ribitol, D-sorbitol, dulcitol, D-mannitol, volemitol, maltitol, isomalt, lactitol and a mix thereof, typically said powdered sugar alcohol is a crystalline sugar alcohol.

In one embodiment, the carbon dioxide source is a linear dialkyl carbonate or a cyclic carbonate with the general formula R1-O—CO—O—R2 wherein said alkyl groups R1 and R2 are each independently selected from the group consisting of C1-C4 alkyl group, benzyl group or a phenyl group, optionally wherein R1 and R2 is covalently linked to form a cyclic carbonate. The carbon dioxide source can be selected in the list of dimethyl carbonate, diethyl carbonate, diphenyl carbonate, dibenzyl carbonate, propylene carbonate, ethylene carbonate, 1,2-butylene carbonate, glycerol carbonate, 4,5-dimethyl-1,3-dioxolan-2-one and the mix thereof.

The five-membered polycycloaliphatic carbonate used can be a five-membered biscycloaliphatic carbonate or a tricycloaliphatic carbonate. Furthermore, the reactional temperature of the invention's process is between 20° C.-250° C., preferably between 30° C.-200° C. The catalyst used is a basic catalyst, preferably an organo-basic-catalyst.

In one particular embodiment, the carbon dioxide source is dimethyl carbonate, the powdered sugar alcohol is a powdered D-sorbitol and the five-membered polycycloaliphatic carbonate is the bis(cyclo-carbonate) (BisCC).

In one embodiment, the ratio of carbon dioxide source/sugar alcohol ranges from 1 to 18, preferably from 2 to 9, more preferably from 3 to 8. The carbon dioxide source can be supplied by continuous feeding.

In one embodiment, the process of the invention further comprises the additional steps of:
precipitating the five-membered polycycloaliphatic carbonate in an aqueous composition;
optionally, washing the precipitated five-membered polycycloaliphatic carbonate obtained; and
recovering the five-membered polycycloaliphatic carbonate by filtrating and/or drying the precipitate obtained.

The invention also concerns a five-membered cycloaliphatic carbonate obtained according to the process of the invention.

The invention finally concerns a process for producing non-isocyanate polyurethane and/or a polycarbonate and/or polyethers comprising the step of
obtaining a five-membered polycycloaliphatic carbonate according to the process of the invention;
mixing said five-membered polycycloaliphatic carbonate with a compound comprising at least one amine function or one alcohol function.

The compound comprising at least one amine function, is selected from the group consisting of a dimer-based diamine having 36 carbon atoms, a polyetherdiamine compound having a molar mass between 200 and 5000 g/mol, a polyethertriamine having a molar mass between 400 and 5000 g/mol, 4,9-dioxa-1,12-dodecanediamine, 1,12-diaminododecane, 1,10-diaminodecane, 1.8-diaminooctane, 1,6-diaminohexane, 1,5-diaminopentane, 1,5-diamino-2-methylpentane, 1,4-diaminobutane, hexamethylene diamine and the mix thereof.

The compound comprising at least one alcohol function is selected from the group consisting of methan-1-ol, ethan-1-ol, propan-1-ol, butan-1-ol, hexan-1-ol, octan-1-ol, decan-1-ol, dodecan-1-ol, 1,3-propandiol, 1,4-butanediol, 1,6-hexanediol, 1,8-octanediol, 1,10-decanediol, 1,12-dodecanediol and the mix thereof.

### DETAILED DESCRIPTION

The invention is about a process for producing a five-membered polycycloaliphatic carbonate comprising a step of reacting a suspension of a powdered sugar alcohol typically a crystalline sugar alcohol with a carbon dioxide source preferably a dimethylcarbonate and a catalyst soluble in said carbon dioxide source at the reactional temperature, preferably at less than 250° C.

According to the invention, the “five-membered polycycloaliphatic carbonate” means a bicyclic or tricyclic carbonate with or without aromatic substituent. Typically, the “five membered polycycloaliphatic carbonate” is a five-membered biscycloaliphatic carbonate or a tricycloaliphatic carbonate of:

general formula A,

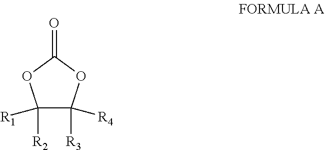

wherein R1, R2, R3 and R4 are each independently of the others;
an hydrogen atom;
a linear and/or branched aliphatic chain having 1 to 18 carbon atoms, preferably 1 to 12 carbon atoms, typically 1 to 10, advantageously, said aliphatic chain is:
   a saturated aliphatic chain;
   an unsaturated aliphatic chain;
   a phenyl group;
   a benzyl group;
   an ethylene carbonate;
   a propylene carbonate;
   a glycerol carbonate;
   a [4,4′-bis(1,3-dioxolane)]-2,2′-dione; or

a group having the general formula B wherein said group is linked by one of the carbon atoms in positions 1, 2, 3 or 4;

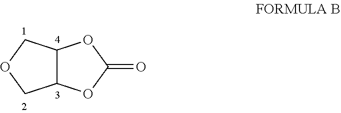

a group having the general formula C wherein said group is linked by one of the carbon atoms in positions 1, 2, 3, 4 or 5;

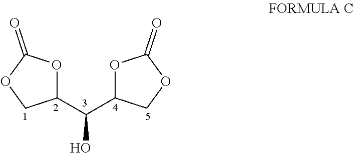

a group having the general formula D wherein said group is linked by one of the carbon atoms in position 1 or 4;

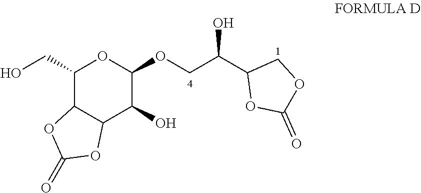

a group having the general formula E wherein said group is linked by one of the carbon atoms in position 1 or 4;

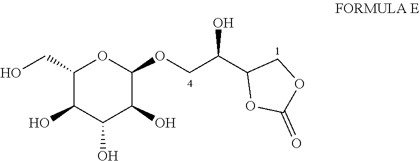

and wherein at least one of R1, R2, R3 and R4 is a five-membered cycloaliphatic carbonate.

Advantageously, the present invention can be carried out without the presence of a solvent, namely carcinogenic, mutagenic or reprotoxic solvents. Typically, the reaction is implemented without any added solvent such as methanol, ethanol, isopropanol, N,N-dimethylformamide, pyridine, trimethylamine, 1,4-dioxane, tetrahydrofuran, toluene or acetone.

“Sugar alcohol” (also referred to as “Hydrogenated sugar”) means a compound obtained by hydrogenation (adding hydrogen) of the reductive end group in sugar. Typically, the process of the invention can be implemented with a powdered sugar alcohol, advantageously a crystalline sugar alcohol. Typically said powdered sugar alcohol is selected among erythritol, arabitol, xylitol, ribitol, D-sorbitol, dulcitol, D-mannitol, volemitol, maltitol, isomalt, lactitol and a mix thereof, preferably erythritol, D-sorbitol, D-mannitol, volemitol and a mix thereof, more preferably D-Sorbitol or D-mannitol.

The “carbon dioxide source” used in the process of the present invention is a linear dialkyl carbonate or a cyclic carbonate with the general formula R1-O—CO—O—R2 wherein said alkyl groups R1 and R2 are each independently selected from the group consisting of C1-C4 alkyl group, benzyl group or a phenyl group, and optionally, wherein R1 and R2 are covalently linked to form a cyclic carbonate.

R1 and R2 can be covalently linked together with the carbon atoms to which R1 and/or R2 are attached, or with their carbon atoms. Typically, the carbon dioxide source is selected in the list of a dimethyl carbonate, a diethyl carbonate, a diphenyl carbonate, dibenzyl carbonate, propylene carbonate, ethylene carbonate, 1,2-butylene carbonate, glycerol carbonate, 4,5-dimethyl-1,3-dioxolan-2-one and the mix thereof, preferably between dimethyl carbonate, diethyl carbonate, diphenyl carbonate, ethylene carbonate, propylene carbonate and the mix thereof and more preferably between dimethyl carbonate, a diethyl carbonate, propylene carbonate and the mix thereof.

Advantageously, the carbon dioxide source is dimethyl carbonate, the powdered sugar alcohol is a powdered D-sorbitol and the five-membered polycycloaliphatic carbonate is the bis(cyclo-carbonate) (BisCC).

The term “alkyl” by itself or as part of another substituent refers to a hydrocarbyl radical of Formula CₙH₂ₙ₊₁ wherein n is a number greater than or equal to 1. Generally, alkyl groups of this invention comprise from 1 to 6 carbon atoms, preferably from 1 to 4 carbon atoms, more preferably from 1 to 3 carbon atoms, still more preferably 1 to 2 carbon atoms. Alkyl groups may be linear or branched and may be substituted as indicated herein. for example, C1-C4 alkyl means an alkyl of one to four carbon atoms. C1-C6 alkyl includes all linear, or branched alkyl groups with between 1 and 6 carbon atoms, and thus includes methyl, ethyl, n-propyl, i-propyl, butyl and its isomers (e.g. n-butyl, i-butyl and t-butyl); pentyl and its isomers, hexyl and its isomers.

The term “catalyst” means a compound that causes or accelerates a chemical reaction without itself being affected. Typically, the catalyst is a basic catalyst preferably an organo-basic-catalyst.

The term “organo-basic-catalyst” means an organic catalyst comprising carbon, hydrogen and any other non-metal element found in organic compounds (heteroatom). In one embodiment, the heteroatom is selected from P, N and O. Typically, the catalyst is basic organo-catalyst preferably selected among thiourea, 4-dimethylaminopyridine, trimethylamine, dimethylcyclohexylamine, 1,1,3,3-tetramethyl guanidine (TMG), 1,5,7-triaza-bicyclo-[4.4.0]dec-5-ene (TBD), 7-methyl-1,5,7-triazabicyclo[4.4.0]dec-5-ene (MTBD), 1,8-Diazabicyclo[5.4.0]undec-7-ene (DBU) and N-tert-butyltris(pyrrolidino)phosphinimine (BTPP), 2-tert-Butylimino-2-diethylamino-1,3-dimethylperhydro-1,3,2-diazaphosphorine. Advantageously, the organo-basic-catalyst are selected among TMG, MTBD, TBD, DBU and BTPP.

In one embodiment, the process for producing a five-membered polycycloaliphatic carbonate comprises a step of reacting:
a suspension of a powdered sugar alcohol, preferably a crystalline sugar alcohol selected from. erythritol, arabitol, xylitol, ribitol, D-sorbitol, dulcitol, D-mannitol, volemitol, maltitol, isomalt, lactitol and a mix thereof;
within a carbon dioxide source is a linear dialkyl carbonate or a cyclic carbonate with the general formula R1-O—CO—O—R2 wherein said alkyl groups R1 and R2 are each independently selected from the group consisting of C1-C4 alkyl group, benzyl group or a phenyl group, optionally wherein R1 and R2 is covalently linked to form a cyclic carbonate; and
a basic catalyst preferably an organo-basic catalyst.

In one embodiment, the process for producing a five-membered polycycloaliphatic carbonate comprises a step of reacting:
a suspension of a powdered sugar alcohol, preferably a crystalline sugar alcohol selected from. erythritol, arabitol, xylitol, ribitol, D-sorbitol, dulcitol, D-mannitol, volemitol, maltitol, isomalt, lactitol and a mix thereof;
within a carbon dioxide source is a linear dialkyl carbonate or a cyclic carbonate selected from a group comprising dimethyl carbonate, diethyl carbonate, diphenyl carbonate, dibenzyl carbonate, propylene carbonate, ethylene carbonate, 1,2-butylene carbonate, glycerol carbonate, 4,5-dimethyl-1,3-dioxolan-2-one and the mix thereof; and
a basic catalyst preferably an organo-basic catalyst selected from a group comprising thiourea, 4-dimethylaminopyridine, trimethylamine, dimethylcyclohexylamine, 1,1,3,3-tetramethyl guanidine (TMG), 1,5,7-triaza-bicyclo-[4.4.0]dec-5-ene (TBD), 7-methyl-1,5,7-triazabicyclo[4.4.0]dec-5-ene (MTBD), 1,8-Diazabicyclo[5.4.0]undec-7-ene (DBU) and N-tert-butyltris(pyrrolidino)phosphinimine (BTPP), 2-tert-Butylimino-2-diethylamino-1,3-dimethylperhydro-1,3,2-diazaphosphorine.

In one embodiment, the process for producing a five-membered polycycloaliphatic carbonate comprises a step of reacting:
a suspension of a powdered sugar alcohol, preferably a crystalline sugar alcohol selected from, arabitol, xylitol, ribitol, D-sorbitol, dulcitol, D-mannitol, volemitol, maltitol, isomalt, lactitol and a mix thereof;
within a carbon dioxide source is a linear dialkyl carbonate or a cyclic carbonate selected from a group comprising dimethyl carbonate, diethyl carbonate, diphenyl carbonate, dibenzyl carbonate, propylene carbonate, ethylene carbonate, 1,2-butylene carbonate, glycerol carbonate, 4,5-dimethyl-1,3-dioxolan-2-one and the mix thereof; and
a basic catalyst preferably an organo-basic catalyst selected from a group comprising thiourea, 4-dimethylaminopyridine, trimethylamine, dimethylcyclohexylamine, 1,1,3,3-tetramethyl guanidine (TMG), 1,5,7-triaza-bicyclo-[4.4.0]dec-5-ene (TBD), 7-methyl-1,5,7-triazabicyclo[4.4.0]dec-5-ene (MTBD), 1,8-Diazabicyclo[5.4.0]undec-7-ene (DBU) and N-tert-butyltris(pyrrolidino)phosphinimine (BTPP), 2-tert-Butylimino-2-diethylamino-1,3-dimethylperhydro-1,3,2-diazaphosphorine.

In one embodiment, the process for producing a five-membered polycycloaliphatic carbonate comprises a step of reacting:
a suspension of a powdered sugar alcohol, preferably a crystalline sugar alcohol selected from. erythritol, xylitol, D-sorbitol, D-mannitol and a mix thereof;
within a carbon dioxide source is a linear dialkyl carbonate or a cyclic carbonate selected from a group comprising dimethyl carbonate, diethyl carbonate, diphenyl carbonate, dibenzyl carbonate, propylene carbonate, ethylene carbonate, 1,2-butylene carbonate, glycerol carbonate, 4,5-dimethyl-1,3-dioxolan-2-one and the mix thereof; and
a basic catalyst preferably an organo-basic catalyst selected from a group comprising thiourea, 4-dimethylaminopyridine, trimethylamine, dimethylcyclohexylamine, 1,1,3,3-tetramethyl guanidine (TMG), 1,5,7-triaza-bicyclo-[4.4.0]dec-5-ene (TBD), 7-methyl-1,5,7-triazabicyclo[4.4.0]dec-5-ene (MTBD), 1,8-Diazabicyclo[5.4.0]undec-7-ene (DBU) and N-tert-butyltris(pyrrolidino)phosphinimine (BTPP), 2-tert-Butylimino-2-diethylamino-1,3-dimethylperhydro-1,3,2-diazaphosphorine.

In one embodiment, the process for producing a five-membered polycycloaliphatic carbonate comprises a step of reacting:
a suspension of a powdered sugar alcohol, preferably a crystalline sugar alcohol selected from. arabitol, xylitol, ribitol, D-sorbitol, dulcitol, volemitol, maltitol, isomalt, lactitol and a mix thereof;
within a carbon dioxide source is a linear dialkyl carbonate or a cyclic carbonate selected from a group comprising dimethyl carbonate, diethyl carbonate, diphenyl carbonate, dibenzyl carbonate, propylene carbonate, ethylene carbonate, 1,2-butylene carbonate, glycerol carbonate, 4,5-dimethyl-1,3-dioxolan-2-one and the mix thereof; and
a basic catalyst preferably an organo-basic catalyst selected from a group comprising thiourea, 4-dimethylaminopyridine, trimethylamine, dimethylcyclohexylamine, 1,1,3,3-tetramethyl guanidine (TMG), 1,5,7-triaza-bicyclo-[4.4.0]dec-5-ene (TBD), 7-methyl-1,5,7-triazabicyclo[4.4.0]dec-5-ene (MTBD), 1,8-Diazabicyclo[5.4.0]undec-7-ene (DBU) and N-tert-butyltris(pyrrolidino)phosphinimine (BTPP), 2-tert-Butylimino-2-diethylamino-1,3-dimethylperhydro-1,3,2-diazaphosphorine.

In one embodiment, the process for producing a five-membered polycycloaliphatic carbonate comprises a step of reacting:
a suspension of a powdered sugar alcohol, preferably a crystalline sugar alcohol selected from. erythritol, arabitol, xylitol, ribitol, D-sorbitol, dulcitol, D-mannitol, volemitol, maltitol, isomalt, lactitol and a mix thereof;
within a carbon dioxide source is a linear dialkyl carbonate or a cyclic carbonate selected from a group comprising dimethyl carbonate, diethyl carbonate, diphenyl carbonate, dibenzyl carbonate, propylene carbonate, ethylene carbonate, 1,2-butylene carbonate, glycerol carbonate, 4,5-dimethyl-1,3-dioxolan-2-one and the mix thereof; and
a basic catalyst preferably an organo-basic catalyst selected from a group comprising 1,1,3,3-tetramethyl guanidine (TMG), 1,5,7-triaza-bicyclo-[4.4.0]dec-5-ene (TBD), 7-methyl-1,5,7-triazabicyclo[4.4.0]dec-5-ene (MTBD), 1,8-Diazabicyclo[5.4.0]undec-7-ene (DBU) and N-tert-butyltris(pyrrolidino)phosphinimine (BTPP).

In one embodiment, the process for producing a five-membered polycycloaliphatic carbonate comprises a step of reacting:
a suspension of a powdered sugar alcohol, preferably a crystalline sugar alcohol selected from. erythritol, arabitol, xylitol, ribitol, D-sorbitol and a mix thereof;
within a carbon dioxide source is a linear dialkyl carbonate or a cyclic carbonate selected from a group comprising dimethyl carbonate, diethyl carbonate, diphenyl carbonate, dibenzyl carbonate, propylene carbonate, ethylene carbonate, 1,2-butylene carbonate, glycerol carbonate, 4,5-dimethyl-1,3-dioxolan-2-one and the mix thereof; and
a basic catalyst preferably an organo-basic catalyst selected from a group comprising 1,1,3,3-tetramethyl guanidine (TMG), 1,5,7-triaza-bicyclo-[4.4.0]dec-5-ene (TBD), 7-methyl-1,5,7-triazabicyclo[4.4.0]dec-5-ene (MTBD), 1,8-Diazabicyclo[5.4.0]undec-7-ene (DBU) and N-tert-butyltris(pyrrolidino)phosphinimine (BTPP).

In one embodiment, the process for producing a five-membered polycycloaliphatic carbonate comprises a step of reacting:
a suspension of a powdered sugar alcohol, preferably a crystalline sugar alcohol selected from. erythritol, arabitol, xylitol, ribitol, D-sorbitol, dulcitol, D-mannitol, volemitol, maltitol, isomalt, lactitol and a mix thereof;
within a carbon dioxide source is a linear dialkyl carbonate or a cyclic carbonate selected from a group comprising dimethyl carbonate, diethyl carbonate, diphenyl carbonate, dibenzyl carbonate, propylene carbonate and a mic thereof; and
a basic catalyst preferably an organo-basic catalyst selected from a group comprising 1,1,3,3-tetramethyl guanidine (TMG), 1,5,7-triaza-bicyclo-[4.4.0]dec-5-ene (TBD), 7-methyl-1,5,7-triazabicyclo[4.4.0]dec-5-ene (MTBD), 1,8-Diazabicyclo[5.4.0]undec-7-ene (DBU) and N-tert-butyltris(pyrrolidino)phosphinimine (BTPP).

In one embodiment, the process for producing a five-membered polycycloaliphatic carbonate comprises a step of reacting:
a suspension of a powdered sugar alcohol, preferably a crystalline sugar alcohol selected from D-sorbitol, D-mannitol, maltitol and a mix thereof;
within a carbon dioxide source is a linear dialkyl carbonate or a cyclic carbonate selected from a group comprising dimethyl carbonate, diethyl carbonate, diphenyl carbonate, dibenzyl carbonate, propylene carbonate, ethylene carbonate, 1,2-butylene carbonate, glycerol carbonate, 4,5-dimethyl-1,3-dioxolan-2-one and the mix thereof; and
a basic catalyst preferably an organo-basic catalyst selected from a group comprising 1,1,3,3-tetramethyl guanidine (TMG), 1,5,7-triaza-bicyclo-[4.4.0]dec-5-ene (TBD), 7-methyl-1,5,7-triazabicyclo[4.4.0]dec-5-ene (MTBD), 1,8-Diazabicyclo[5.4.0]undec-7-ene (DBU) and N-tert-butyltris(pyrrolidino)phosphinimine (BTPP).

In one embodiment, the process for producing a five-membered polycycloaliphatic carbonate comprises a step of reacting:
a suspension of a powdered sugar alcohol, preferably a crystalline sugar alcohol selected from D-sorbitol, D-mannitol, maltitol and a mix thereof;
within dimethyl carbonate; and
a basic catalyst preferably an organo-basic catalyst selected from a group comprising 1,1,3,3-tetramethyl guanidine (TMG), 1,5,7-triaza-bicyclo-[4.4.0]dec-5-ene (TBD), 7-methyl-1,5,7-triazabicyclo[4.4.0]dec-5-ene (MTBD), 1,8-Diazabicyclo[5.4.0]undec-7-ene (DBU) and N-tert-butyltris(pyrrolidino)phosphinimine (BTPP).

In one embodiment, the process for producing a five-membered polycycloaliphatic carbonate comprises a step of reacting:
a suspension of powdered D-sorbitol sugar alcohol, preferably a crystalline D-sorbitol;
with dimethyl carbonate; and
a basic catalyst preferably an organo-basic catalyst selected from a group comprising 1,1,3,3-tetramethyl guanidine (TMG), 1,5,7-triaza-bicyclo-[4.4.0]dec-5-ene (TBD), 7-methyl-1,5,7-triazabicyclo[4.4.0]dec-5-ene (MTBD), 1,8-Diazabicyclo[5.4.0]undec-7-ene (DBU) and N-tert-butyltris(pyrrolidino)phosphinimine (BTPP).

According to the invention, said process is implemented at the needed temperature to solubilize the catalyst, typically at a temperature range between 20-250° C., preferably 30-200° C., more preferably 35-150° C. or 35-140° C. In one embodiment, the temperature ranges from 75° to 150° C. In one embodiment, the temperature ranges from 30° to 150° C. In one embodiment, the temperature ranges from 30° to 100° C. In one embodiment, the temperature ranges from 35° to 80° C. Advantageously, said process is implemented at a temperature between 37 and 100° C. or 40 and 90° C., for example, between 45-80° C.

According to the invention, the catalyst compound is soluble in said carbon dioxide source at the reactional temperature. Advantageously, when the carbon dioxide source is dimethyl carbonate, the catalyst compound is soluble at temperature between 45-80° C. Such catalyst is for example the TBD, DBU or BTPP.

According to one embodiment, said process comprises a precipitation step of the five-membered polycycloaliphatic carbonate in an aqueous composition such as water, typically at ambient temperature.

Optionally, a washing step of the precipitated five-membered polycycloaliphatic carbonate is envisaged.

Then the precipitated five-membered polycycloaliphatic carbonate (with or without a washing step) is recovered by a filtration step and/or a drying step.

Typically, the carbon dioxide source is added by continuous feeding. The continuous feed is particularly advantageous in that it increases the global yield of the reaction by reducing the probability of side product formation and diminishing the potential azeotrope formation between the carbon dioxide source and the natural subsidiary product of the reaction such as methanol for example depending on the carbon dioxide source.

Typically, the ratio carbon dioxide source/sugar alcohol is between 1 to 18, preferably between 2 to 9 or 2 to 8, more preferably between 3 to 8 or 3 to 7. Advantageously the ratio carbon dioxide source/sugar alcohol is between 4 and 7. Advantageously the ratio carbon dioxide source/sugar alcohol ranges from 4 to 7, from 5 to 7, from 4 to 6 or from 6 to 7.

The invention also concerns a five-membered polycycloaliphatic carbonate obtained according to the process of the invention.

Such five-membered polycycloaliphatic carbonate are particularly advantageous as monomer in production of non-isocyanate polyurethane (also called polyhydroxyurethane) and/or a polycarbonate and/or polyethers.

The invention is also about a process for producing non-isocyanate polyurethane and/or a polycarbonate and/or polyethers comprising the step of:
a) obtaining a five-membered polycycloaliphatic carbonate according to the process of the invention;
b) mixing said five-membered polycycloaliphatic carbonate with;
   a compound comprising at least one amine function, typically a diamine, triamine, tetramine, pentamine function or a mix thereof, preferably said compound is selected from the group consisting of a dimer-based diamine having 36 carbon atoms , a polyetherdiamine compound having a molar mass between 200 and 5000 g/mol, a polyethertriamine having a molar mass between 400 and 5000 g/mol , 4,9-dioxa-1,12-dodecanediamine, 1,12-diaminododecane, 1,10-diaminodecane, 1.8-diaminooctane, 1,6-diaminohexane, 1,5-diaminopentane, 1,5-diamino-2-methylpentane, 1,4-diaminobutane, hexamethylene diamine and the mix thereof, for obtaining a non-isocyanate polyurethane; or
   a compound comprising at least one alcohol function, said compound is preferably selected from the group consisting of methan-1-ol, ethan-1-ol, propan-1-ol, butan-1-ol, hexan-1-ol, octan-1-ol, decan-1-ol, dodecan-1-ol, 1,3-propandiol, 1,4-butanediol, 1,6-hexanediol, 1,8-octanediol, 1,10-decanediol, 1,12-dodecanediol and the mix thereof, for obtaining a polycarbonate and/or a polyether.

In one embodiment, the compound comprising at least one amine function, is selected from the group consisting of a dimer-based diamine having 36 carbon atoms , a polyetherdiamine compound having a molar mass between 200 and 5000 g/mol, a polyethertriamine having a molar mass between 400 and 5000 g/mol , 4,9-dioxa-1,12-dodecanediamine, 1,12-diaminododecane, 1,10-diaminodecane, 1.8-diaminooctane, 1,6-diaminohexane, 1,5-diaminopentane, 1,5-diamino-2-methylpentane, 1,4-diaminobutane, hexamethylene diamine and the mix thereof.

In one embodiment, the compound comprising at least one amine function, is selected from the group consisting of a dimer-based diamine having 36 carbon atoms, a polyetherdiamine compound having a molar mass between 200 and 5000 g/mol, a polyethertriamine having a molar mass between 400 and 5000 g/mol and the mix thereof.

In one embodiment, the compound comprising at least one amine function, is selected from the group consisting of 4,9-dioxa-1,12-dodecanediamine, 1,12-diaminododecane, 1,10-diaminodecane, 1.8-diaminooctane, 1,6-diaminohexane, 1,5-diaminopentane, 1,5-diamino-2-methylpentane, 1,4-diaminobutane, hexamethylene diamine and the mix thereof.

In one embodiment, the compound comprising at least one amine function, is selected from the group consisting of a dimer-based diamine having 36 carbon atoms, preferably a fatty acid dimer-based diamine having 36 carbon atoms 4,9-dioxa-1,12-dodecanediamine, 1,12-diaminododecane, 1,10-diaminodecane, 1.8-diaminooctane, 1,6-diaminohexane, 1,5-diaminopentane, 1,5-diamino-2-methylpentane, 1,4-diaminobutane, hexamethylene diamine and the mix thereof.

In one embodiment, the compound comprising at least one amine function, is selected from the group consisting of a dimer-based diamine having 36 carbon atoms, preferably a fatty acid dimer-based diamine having 36 carbon atoms, 4,9-dioxa-1,12-dodecanediamine, 1,12-diaminododecane, 1,10-diaminodecane, 1.8-diaminooctane, 1,6-diaminohexane, 1,5-diaminopentane, 1,5-diamino-2-methylpentane, 1,4-diaminobutane, hexamethylene diamine and the mix thereof.

In one embodiment, the compound comprising at least one amine function, is selected from the group consisting of a dimer-based diamine having 36 carbon atoms, preferably a fatty acid dimer-based diamine having 36 carbon atoms, 4,9-dioxa-1,12-dodecanediamine, 1,12-diaminododecane, 1,10-diaminodecane, 1.8-diaminooctane, 1,6-diaminohexane, 1,5-diaminopentane, 1,5-diamino-2-methylpentane, 1,4-diaminobutane, hexamethylene diamine and the mix thereof.

In one embodiment, the compound comprising at least one amine function, is selected from the group consisting of a dimer-based diamine having 36 carbon atoms, preferably a fatty acid dimer-based diamine having 36 carbon atoms, 1,5-diamino-2-methylpentane, 1,4-diaminobutane, hexamethylene diamine and the mix thereof.

In one embodiment, the compound comprising at least one amine function, is selected from the group consisting of a dimer-based diamine having 36 carbon atoms, preferably a fatty acid dimer-based diamine having 36 carbon atoms, 1,5-diamino-2-methylpentane, and the mix thereof.

In one embodiment, the compound comprising at least one amine function, is selected from the group consisting of a dimer-based diamine having 36 carbon atoms, preferably a fatty acid dimer-based diamine having 36 carbon atoms, 1,4-diaminobutane, hexamethylene diamine and the mix thereof.

In one embodiment, the compound comprising at least one amine function, is selected from the group consisting of 1,5-diamino-2-methylpentane, 1,4-diaminobutane, hexamethylene diamine and the mix thereof.

In one embodiment, the compound comprising at least one amine function is 1,5-diamino-2-methylpentane.

In one embodiment, the compound comprising at least one amine function is 1,4-diaminobutane.

In one embodiment, the compound comprising at least one amine function is hexamethylene diamine

In one embodiment, the compound comprising at least one alcohol function, said compound is preferably selected from the group consisting of methan-1-ol, ethan-1-ol, propan-1-ol, butan-1-ol, hexan-1-ol, octan-1-ol, decan-1-ol, dodecan-1-ol, 1,3-propandiol, 1,4-butanediol, 1,6-hexanediol, 1,8-octanediol, 1,10-decanediol, 1,12-dodecanediol and the mix thereof.

In one embodiment, the compound comprising at least one alcohol function, said compound is preferably selected from the group consisting of butan-1-ol, hexan-1-ol, octan-1-ol, decan-1-ol, dodecan-1-ol, 1,3-propandiol, 1,4-butanediol, 1,6-hexanediol, 1,8-octanediol, 1,10-decanediol, 1,12-dodecanediol and the mix thereof.

In one embodiment, the compound comprising at least one alcohol function, said compound is preferably selected from the group consisting of methan-1-ol, ethan-1-ol, propan-1-ol, butan-1-ol, hexan-1-ol, octan-1-ol, decan-1-ol, dodecan-1-ol, 1,3-propandiol, 1,4-butanediol, 1,6-hexanediol, 1,8-octanediol, and the mix thereof.

In one embodiment, the compound comprising at least one alcohol function, said compound is preferably selected from the group consisting of octan-1-ol, decan-1-ol, dodecan-1-ol, 1,6-hexanediol, 1,8-octanediol, 1,10-decanediol, 1,12-dodecanediol and the mix thereof.

In one embodiment, the compound comprising at least one alcohol function, said compound is preferably selected from the group consisting of octan-1-ol, decan-1-ol, dodecan-1-ol, 1,10-decanediol, 1,12-dodecanediol and the mix thereof.

In one embodiment, the compound comprising at least one alcohol function, said compound is preferably selected from the group consisting of octan-1-ol, decan-1-ol, dodecan-1-ol, and the mix thereof.

In one embodiment, the compound comprising at least one alcohol function, said compound is preferably selected from the group consisting of octan-1-ol, decan-1-ol, dodecan-1-ol, and the mix thereof.

In one embodiment, the compound comprising at least one alcohol function is octan-1-ol.

A non-isocyanate polyurethane (NIPU) is an oligomer or a polymer containing urethane function and obtained without isocyanate-based monomers. Such urethanes functions are mainly obtained through aminolysis by reacting an amine function with a cyclic carbonate function, or transurethanification, or acyl azid AB type autocondensation. Using aminolysis process, these polymers are generally obtained by working in stoichiometric ratio or close to a stoichiometric ratio of amine and cyclic carbonate functions. Such NIPU are also called polyhydroxyurethane due to the formation of an hydroxyl group when the amine function opens the cyclic carbonate. This hydroxyl group can be primary or secondary hydroxyl group according to the chemical structure of the cyclic carbonate and always in beta position of the urethane function.

The invention is also about a method of preparing a NIPU foam, comprising the steps of:
a) obtaining a five-membered polycycloaliphatic carbonate according to the process of the invention;
b) mixing said five-membered polycycloaliphatic carbonate with a compound comprising at least one amine function;
c) adding a blowing agent.

A non-isocyanate polyurethane (NIPU) foam is an alveolar material obtained from blown NIPU. To blow a NIPU composition, a blowing agent is added to the composition before NIPU polymerization or full polymerization. Such foam can be rigid or soft with closed or opened cells.

A blowing agent refers to a compound which induces a chemical or physical expansion when added into a composition. Typically, the blowing agent is chosen among pentane isomers, hydrocarbons, liquid hydrofluorocarbons or liquid hydrofluorocarbon blends, CO₂, air or a mixture thereof.

The invention is also about a process for producing a non-isocyanate polyurethane coatings, elastomeric materials, or NIPU-based adhesives comprising the steps of:
a) obtaining a five-membered polycycloaliphatic carbonate according to the process of the invention;
b) mixing said five-membered polycycloaliphatic carbonate with at least one compound comprising at least one amine function, typically a monoamine, a diamine, a triamine or a mix thereof;
c) optionally adding a pigmentary paste such as azoic pigment;
d) optionally adding 0-5% wt additives like rheological additive or functional additive such as anti-fungal, defoamer or the mix thereof.

The invention is also about a process for producing a polycarbonate and/or a polyether coatings, elastomeric materials, or adhesive comprising the steps of:
a) obtaining a five-membered polycycloaliphatic carbonate according to the process of the invention;
b) mixing said five-membered polycycloaliphatic carbonate with at least one compound comprising at least one alcohol function;
c) optionally adding a pigmentary paste such as azoic pigment;
d) optionally adding 0-5% wt additives like rheological additive or functional additive such as anti-fungal, defoamer or the mix thereof.

The invention is also about the use of a five-membered cycloaliphatic carbonate produced by the process according to the invention

Wherever the phrase “for example”, “such as”, “including” and the like are used herein, the phrase “and without limitation” is understood to follow unless explicitly stated otherwise.

As used in the specification and the appended claims, the singular forms “a”, “an” and “the” include plural referents unless the context clearly dictates otherwise. Thus, for example, reference to “a purified monomer” includes mixtures of two or more purified monomers. The term “comprising” as used herein is synonymous with “including”, “containing”, or “characterized by”, and is inclusive or open-ended and does not exclude additional, unrecited elements or method steps.

“Optional” or “optionally” means that the subsequently described event or circumstance may or may not occur, and that the description includes instances where said event or circumstance occurs and instances where it does not.

Although having distinct meanings, the terms “comprising”, “having”, “containing” and “consisting of” may be replaced with one another throughout the above description of the invention.

The invention will be further illustrated in view of the following examples and figures.

### BRIEF DESCRIPTION OF THE DRAWINGS

FIG. 1 presents the ¹H-NMR and ¹³C-NMR spectra of five-membered BisCC from D-sorbitol.

FIG. 2 is a graph showing the influence of the number of equivalent of DMC on the kinetic profile with distillation bridge device.

FIG. 3 is a graph showing the influence of the number of equivalent of DMC on the kinetic profile with vigreux distillation column.

FIG. 4 presents the ¹H-NMR spectrum of the reagent.

FIG. 5 presents the ¹H-NMR spectrum of the crude synthesis product.

FIG. 6 presents the ¹³C-NMR spectrum of the crude synthesis product.

### EXAMPLES

The present invention is further illustrated by the following examples.

### Example 1: Catalyzed D-Sorbitol Based Bis-Cyclocarbonate Synthesis

Round-bottom flask equipped with a distillation bridge or vigreux fractionating columns or reflux devise was charged one molar equivalent of D-sorbitol, 5% molar equivalent toward D-sorbitol of catalyst choose between TBD, DBU, BTPP, KOH, NaOH, K₂CO₃, TTIP, TNBT or stannous octoate (Sn(oct)) and different amounts of DMC comprise between 3.5 to 18 molars equivalent toward D-sorbitol. The mixture was stirred and heat to 75° C. for 16 to 48 h (table 2). At the end, the media was cooled down at room temperature and 10 mL of distilled water were added to precipitate the product. Reaction products were filtrated over 0.45 μm polyvinylidene fluoride (PVDF) membrane and washed with distilled water then dried under vacuum at 50° C. overnight. One reaction per point was made to calculate product yield for kinetic study. In order to have a constant DMC flow rate and to continuously feed the reaction, a syringe driver was used (Fischer Scientific No 9034914) (table 1).

TABLE 1
Synthesis of BisCC from D-sorbitol
using a continuous feed of DMC

| | | Additional feed | | | |
|---|---|---|---|---|---|
| | Initial feed | Molar | Flow rate | Reaction total | Yield |
| Entry | (molar ratio) | ratio | (mL/h) | time (h) | (%) |
| 1 | 3.35^{a} | 3.35 | 0.15 | 16 | 50 |
| 2 | 3.35^{a} | 6.7 | 0.3 | 16 | 43 |
| 3 | 1^{a} | 6.7 | 0.3 | 16 | 35 |

^{a}DMC

### Example 2: Uncatalyzed D-Sorbitol Based Bis-Cyclocarbonate Synthesis

A 50 mL round-bottom flask equipped with a distillation bridge was charged with one molar equivalent of D-sorbitol and four molar equivalents of EC. The mixture was stirred, heated to 150° C. and maintained under control vacuum of 23-33 mbar to remove sub-reaction product (i.e. Ethylene glycol). To follow the reaction kinetic, this reaction has been made four times for 6 h, 14.5 h, 24 h and 48 h. At the end a brown mixture was obtained. The media was cooled down to room temperature and brought back to atmospheric pressure. 10 mL of ethanol were added and the mixture was heated until full dilution in ethanol. The mixture was then poured into a beaker and let two days at 4° C. for crystallization of the corresponding product. Brown crystals are obtained and recovered by filtration. These brown crystals were finally washed with glacial acetic acid to obtain white crystal.

### Example 3: Polyols Polyethers Synthesis

A 50 mL round flask was charged with different molar ratio of D-sorbitol based bis-cyclocarbonate and 1-octanol at different temperatures in a range of 90° C. to 150° C. for 24 h with 0.7 or 5 mol % toward bis-cyclocarbonate of K2CO3 as catalyst (Table 3). 1 mL of dimethylsufoxide was added as solvent then the flask was closed with septum, flush under argon for 10 minutes before launching. During all the reactions, the septum was pierced with a needle to evacuate gaseous subsidiaries products (sub-product) induced by reactions. The end reaction media was a dark product. In main cases, the product obtained was liquid and thus precipitated in 50 mL of toluene, when solid, the product was washed in 50 mL of water.

### Example 4: Non-Isocyanate Polyurethane Synthesis

A 50 mL round flask was charged with different diamine chosen between: 1,4 diminobutane (1,4 B), 1,2-diamino-2-methylpentane (1,5 MD), priamine 1075 (P1075) and 1,6-diaminohexane (1,6 H) and the equivalent molar ratio of D-sorbitol based bis-cyclocarbonate. Then two blending compositions were study with two diamines per NIPU: P1075/1,4 B or P1075/1,5 MD in four ratios such as 80/20, 60/40, 40/60 and 20/80, respectively. Methanol was added as reaction solvent for each synthesis. The media was heat to reflux temperature (66° C.) for 72 h then the methanol was evaporated. The viscous product was poor on a non-adhesive sheet to be hot pressed or placed into an oven at 80° C. or 72 additional hours before being hot press at 80° C. to obtain material plates.

### Example 5: Bis-Cyclocarbonate Characterization

The reaction product of each reaction was a white powder directly obtained by precipitation in water for Entries 8 to 19 and after re-crystallization for Entry 1.

Tₘ=214° C.

Elementar analysis: C, 44.6%; H, 3.88%; N, 0%.

ESI: [M+K⁺]=254.99 g·mol⁻¹

FTIR (ATR); υ (cm⁻¹)=2995−2992 (C—H2), 2880 (CH), 1778 (C═O, five-membered cyclic carbonate), 1130 (C—O—C).

¹H-NMR, δ (ppm): 5.4 (m, 2H, CH—CH(O)), 5.0 (m,1H, CHO), 4.6(t, 1H, CH₂O), 4.5(m, H, CH₂O), 4.2(d, 1H,CH₂O), 4.0 (d, 1H,CHO), 3.8 (d, 1H, CH₂O).

¹³C-NMR, δ (ppm): 154.7 (C═O), 153.9 (C═O), 81.1 (CH—O), 80.8 (CH—CHOC(O)), 79.6 (CH2-CHO—C(O)), 74 (CHO—C(O)—CH2O), 71.8 (CH2O), 66.3 (CH2O).

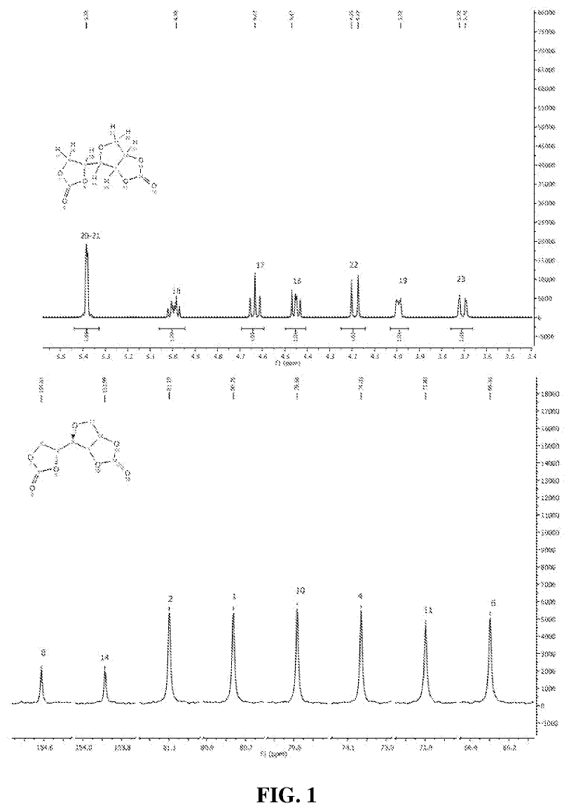

1H-NMR and 13C-NMR peaks (see FIG. 1) are fully attributed in Formula 1.

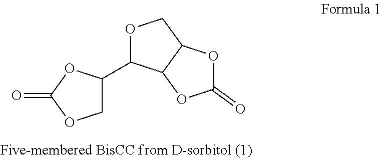 [SMILES: O=C1OCC(C2OCC3OC(=O)OC32)O1]

TABLE 2
Synthesis of BisCC from D-sorbitol and DMC
with different conditions and catalysts

| | Reactional | D-sorbitol/carbonate | | Reaction | Product | Yield |
|---|---|---|---|---|---|---|
| Entry | device | molar ratio | Catalyst | time (h) | obtained | (%) |
| 1 | Distill. Bridge | 4^{a} | None | 14.5 | 1 | 18 |
| 2 | Distill. Bridge | 3.5^{b} | TTIP | 16 | nc | 0 |
| 3 | | 3.5^{b} | TNBT | 16 | nc | 0 |
| 4 | | 3.5^{b} | Sn(oct) | 16 | nc | 0 |
| 5 | | 3.5^{b} | KOH | 16 | nc | 0 |
| 6 | | 3.5^{b} | NaOH | 16 | nc | 0 |
| 7 | | 3.5^{b} | K₂CO₃ | 16 | nc | 0 |
| 8 | | 3.5^{b} | BTPP | 1-16 | 1 | 21 |
| 9 | | 3.5^{b} | DBU | 1-16 | 1 | 24 |
| 10 | | 3.5^{b} | TBD | 1-16 | 1 | 22 |
| 11 | | 7 | BTPP | 16 | 1 | 35 |
| 12 | | 9 | BTPP | 1-16 | 1 | 42 |
| 13 | | 5^{b} | TBD | 1-16 | 1 | 40 |
| 14 | | 7^{b} | TBD | 1-16 | 1 | 41 |
| 15 | Reflux | 3.5^{b} | TBD | 16 | 1 | 10 |
| 16 | Vigreux. | 3.5^{b} | TBD | 16 | 1 | 15 |
| 17 | | 5 | TBD | 16 | 1 | 14 |
| 18 | | 7^{b} | TBD | 1-24 | 1 | 36 |
| 19 | | 9^{b} | TBD | 1-48 | 1 | 45 |
| 20 | | 12^{b} | TBD | 16 | 1&2 | —^{c} |
| 21 | | 15^{b} | TBD | 16 | 1&2 | —^{c} |
| 22 | | 18^{b} | TBD | 16 | 1&2 | —^{c} |

^{a}EC, under vacuum reaction
^{b}DMC
^{c}Obtain product were not pure according to said process and need further purification

According to all characterization data, the obtained product at then end of the reaction is a five-membered BisCC (formula 1). It is the sole product which has been identified and shown after precipitation in water for all reaction except Entry 1 which needed recrystallization in glacial acetic.

### Example 6: Catalyst Efficiency and Screening

First of all, based on Hajiime Komura and al. (BCSJ, vol. 46, 550-553, 1973) work on the production of tris-cyclocarbonate from D-mannitol, a trial for producing tris-cyclocarbonate from D-sorbitol without any solvent, Entry 1 (Table 1) was done in similar conditions. These harsh conditions (high temperature, control vacuum) permitted to provide the right product. Nevertheless, the yield (18%) was far away from the one observed on D-mannitol (65% yield). D-mannitol and D-sorbitol are very similar molecules, only their stereochemistry is changing on one asymmetric carbon (formula 2).

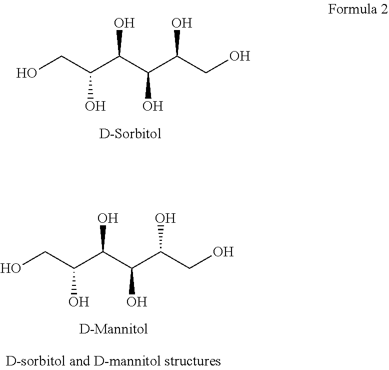 [SMILES: OC[C@@H](O)[C@@H](O)[C@H](O)[C@@H](O)CO ; OC[C@@H](O)[C@@H](O)[C@H](O)[C@H](O)CO]

In more recent work²¹ it has been demonstrated that the difference of reactivity of diols toward carbonates is highly linked to their stereochemistry. Indeed, the trans configuration is not suitable for the formation of five-membered cyclic carbonates because of the ring strength of such cycle and leads to linear carbonates. Besides, diols in cis configuration make possible the cyclization according to previously reported reactional pathway²¹ of D-sorbitol with DMC passing through two possible reactional intermediates: 1,4 sorbitan and the 3,6 sorbitan (formula 3), respectively.

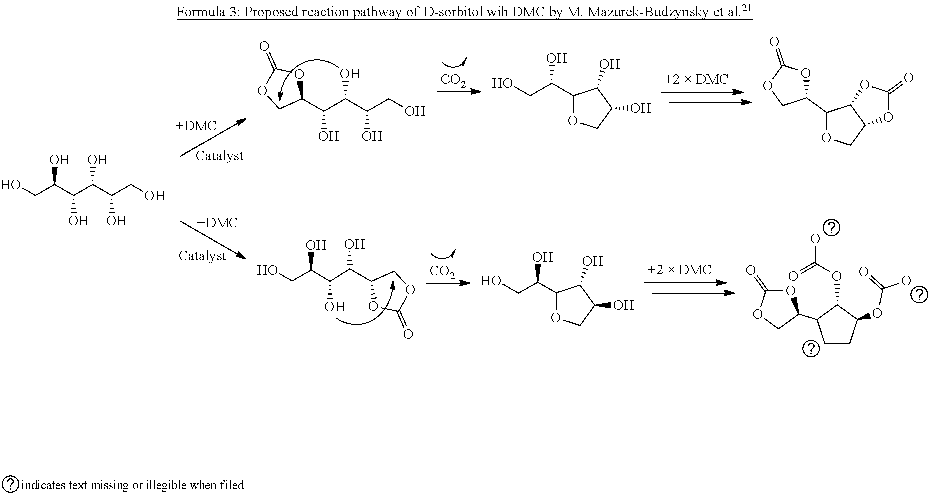

To evaluate TTIP, TNBT, Sn(oct) catalyst performance, three reactions were conducted for 16 h with a distillation bridge at 75° C. (Table 2, Entries 2-4). As the reaction is based on a transesterification mechanism between DMC and D-sorbitol, the first studied catalyst family was metallic-based catalysts which are widely used in this kind of reaction with polymer synthesis and polymer transesterification. All these reactions gave a yield superior to 0%. It is easily assumed that the reaction temperature is too low to activate metallic catalysts which are generally used in a temperature range of 150-250° C.^{22,23}.

Then two catalyst families of strong basis were studied: mineral-based (Table 2, Entries 5-7) and organic-based ones (Table 2, Entry 8-10). These latter are from the phosphazene family, BTPP which is the strongest basis here, (Table 2, Entry 8) with a pK_{BH+}=28.89²⁴, and two strong bases from the guanidine family TBD and DBU (Table 2, Entries 9-10), with pKa=15 and pKa=13.5, respectively. BTPP give similar results than DBU and TBD in the range of 20-25% yield, which is quite odd as we can expect that the strongest basis will increase the reaction rate and the yield. In fact, it has been hypothesized that BTPP is too strong and then increases the formation of other products, instead of the desired one. In particular, it has been assumed that carbonate rings are deprotonaded to create a novel equilibrium between carbonate rings and linear ones. It was completely unexpected to obtain 0% of yield with all tested strong mineral basis as Mazurek-Budzyńska and al.²¹ performed similar reactions in methanol as solvent and obtained 41% of D-sorbitol conversion with K₂CO₃ as catalyst. This major difference comes from the fact that the reaction was implemented without solvent which implies a heterogeneous media with a solid-liquid reaction and not an only liquid one. Assuming this, we checked the solubility of our different catalysts at room temperature and 75° C. It appears that all the mineral catalysts were insoluble in DMC at both temperature. Instead of this TBD, DBU and BTPP were soluble in DMC at 75° C. According to this solubility result we confirmed deprotonation of D-sorbitol in tri-phasic media to induce the trans-esterification reaction between D-sorbitol and DMC with mineral basis is not suitable whereas in the case of real bi-phasic media it allows the catalyst activity by significantly increasing the meeting probability between D-sorbitol hydroxyl group and the catalyst, permitting the deprotonation reaction for the nucleophilic attack on the DMC.

We can conclude this part on catalyst efficiency that strong organo-catalysts are the most appropriate catalysts to perform this kind of reaction. We can also highlight the fact that working in greener ways i.e. in reactive solvent with bi-phasic media imply a good choice of the catalyst to initiate the reaction and to increase yield.

### Example 7: Kinetic and Reactional System Study

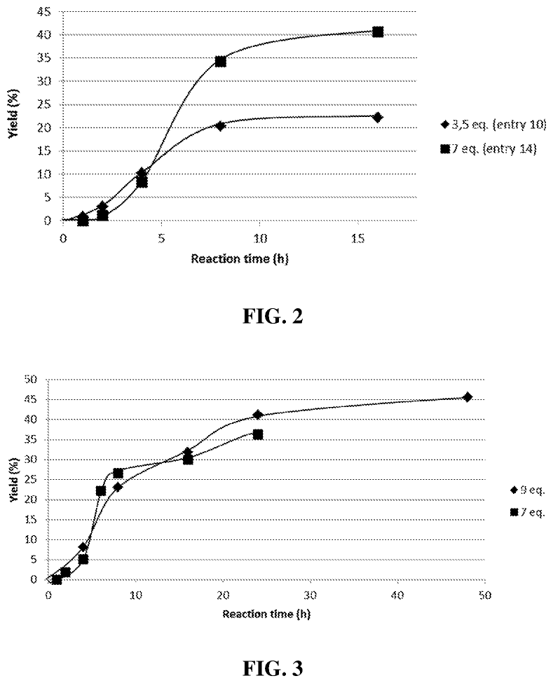

According to previous results, we decided to exclusively worked with organo-catalysts, more precisely TBD-based catalyzed system since it presents a low toxicity and gives similar results in yield as DBU or BTPP. The kinetic profile of the reaction in Entries 10 and 14 (Table 2) were studied at 1, 2, 4, 8 and 16 h of reaction (FIG. 2).

With 3.5 eq. of DMC a plateau at 20% of yield after 8 h of reaction was clearly noticed. With 7 eq. of DMC the same phenomenon is visible after 8 h but the obtained yield is 41%, in this case. Adding more DMC equivalent to the media seems to be an attractive option but it is theoretically not needed as the reactional mechanism implies 3 equivalent (Formula 3) of DMC but it has been noticed that the reactional media was dried after 16 h of reaction meaning a loss of reactant. By 1H-NMR analysis of the distillate DMC and methanol mixture was found indicating the formation of an azeotrope between the normal sub-product of the reaction and the reactant. This azeotrope has been reported by John H. Clements²⁵, the azeotrope molar ratio DMC/MeOH is closed to 30/70. Nevertheless, this does not explain the dry aspect of the media, the evaporation of DMC by vapor tensor cannot be excluded. To control this, two main options are available. The first one is to increase the quantity of DMC toward D-sorbitol. Doing this, an optimum at 5 eq. of DMC was found which conducts to 40% of yield (Table 2, Entry 13). With more equivalent of DMC i.e. 7 eq., the yield only increase of 1% (Table 2, Entry 14) compared to the reaction performed with 5 eq. which is insignificant. According to previous observations, we preferably work with 5 eq. of DMC when distillation bridge device is used. To increase the reaction global yield, the second option was to change the reactional device by a reflux system assuming that the DMC reflux will be sufficient to push the reaction toward products by maintaining subsidiary product i.e. methanol at gaseous state inside the reflux column and in minority in the media. Another option was to use a vigreux fractionating column to break the aezeotrope to condense the DMC and evacuate the methanol.

Both options were implemented (Table 2, Entry 15, 16) and it clearly appears that reflux device was not suitable as the yield was only 10% against 15% with the vigreux fractionating column. In similar conditions with distillation bridge (Table 1, Entry 10) a yield of 22% was obtained. Like previously, with vigreux fractionating column, the number of equivalent of DMC was increased to 5, 7 and 9 (Table 2, Entries 17-19) allowing to increase the reaction time up to 48 h with 9 éq. DMC with a final yield of 14%, 36% and 45%, respectively. The kinetic profile of the reaction with 9 eq. DMC was similar to the previous one, reaching a plateau between 24 h and 48 h of reaction (FIG. 3). In the view of the obtained results (Table 2, Entry 17-19), the assumption of breaking the azeotrope between DMC and the reaction subsidiary product (methanol) by using a vigreux fractionating column is not confirmed. Indeed, a remaining amount of DMC is still distillated with the methanol and the reaction does not shift in favor of the BisCC as the measured global yield only increases of 5%. Nevertheless, it is possible on larger scale (i.e., industrial case) that the use of fractionating distillation device to separate DMC of the methanol and then to re-introduce DMC in the reactional media and to eliminate the corresponding methanol is a way to increase the reaction yield by displacing the reaction equilibrium toward the synthesis of the selected products.

To push further the yield, the number of DMC equivalent was increased (Table 2, Entries 20-22) but some impurities were observed in the obtained products. The main hypothesis was that the high availability of DMC in the media was favoring the formation of “branched D-sorbitol” with linear carbonate instead of carbonate cyclization reactions. According to previous observations, we preferably work with 9 eq. of DMC when vigreux fractionating column device is used.

This study shows that an optimum yield at 40% can be reach using 5 eq. of DMC, TBD as catalyst with a distillation bridge device during 16 h. Besides, a maximum yield of 45% can be reach using vigreux fractionating column, 9 eq. of DMC in 48 h. These are promising results since the proposed reactional mechanism seems to give a maximum yield of 50% as both 1,4 sorbitan and 3,6 sorbitan seem to have the same probability of formation. A yield of 40% in 16 h seems to be the more efficient result in these solvent free and low temperature conditions.

In the view of the observed results, best conditions to perform this reaction is 5 eq. DMC with a distillation bridge. Vigreux fractionating column gives similar result but the time needed to implement the reaction is three times superior compared to the reaction performed with distillation bridge.

### Example 8: Yield Optimization by Continuous Flow Rate

According to the previously described mechanism, it seems that a yield of 50% can be reached. Based on the previous results, a distillation bridge was used and the initial media (TBD catalyzed) was feed with a continuous flow of DMC to avoid the loss of reactant through the azeotrope with methanol, based on a 16 h reaction. The reaction was initiated using 1 eq. DMC and then a feeding of 6.7 eq. of DMC was performed to always have defiance in DMC to lead the reaction to the formation of bis-cyclocarbonate (Table 1, Entry 3) by promoting carbonate cyclisation. This reaction gave poor yield, probably due to the lack of reactive solvent in the media with low homogenization of the mixture, diminishing the probability of interaction of the different reactants. To verify this hypothesis the reaction was started with 3.5 eq. of DMC and feed 6.7 more eq. of DMC (Table 1, Entry 2). Using this protocol, an increase of the global yield of only 8% was observed. It has been finally found an initial feed of 3.5 eq. of DMC and continuously feed 3.5 more eq. DMC was the optimized conditions with this reactional setting (Table 1, Entry 1) leading to the target yield of 50%. Feeding the reactional media with the right amount of DMC is a suitable way to optimize reactional yield up to 50% of sorbitol conversion. The continuous feed as many advantages. First the reactant quantity in the media is as low as possible which limit sides reactions like the formation of linear carbonate on D-sorbitol hydroxyl mainly privilege by high availability of DMC. It is also a means to limit the azeotrope formation between DMC and methanol as the balance between DMC and methanol in the media is lower.

### Example 9: Ring Opening Polymerization of D-Sorbitol Based Five-Membered Bis-Cyclocarbonate

TABLE 3
Synthesis of polyether/polycarbonate from five-
membered BisCC (1). The Catalyst is K₂CO₃.

| | | Initiator/BisCC (1) | Catalyst | T | Time |
|---|---|---|---|---|---|
| Entry | Initiator | Molar ratio | (% mol) | (° C.) | (h) |
| 1 | 1-octanol | 1/1 | 5 | 150 | 2 |
| 2 | 1-octanol | 1/100 | 5 | 150 | 17 |
| 3 | 1-octanol | 1/50 | 5 | 150 | 24 |
| 4 | 1-octanol | 1/25 | 5 | 150 | 17 |
| 5 | 1-octanol | 1/100 | 0.7 | 150 | 2 |
| 6 | 1-octanol | 1/100 | 0.7 | 150 | 24 |
| 7 | 1-octanol | 1/50 | 0.7 | 150 | 24 |
| 8 | 1-octanol | 1/25 | 0.7 | 150 | 24 |
| 9 | 1-octanol | 1/10 | 0.7 | 150 | 24 |

For all reactions between 1-octanol and the BisCC, a gas release was observed. It is a strong indication that the reaction went according to the general mechanism of the Formula 4.

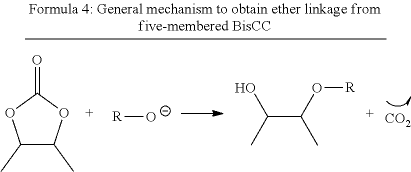

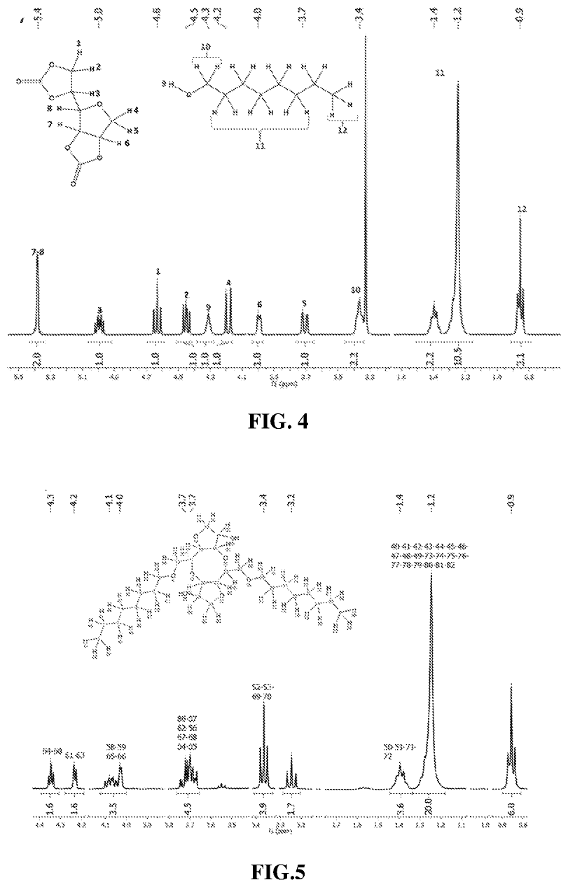

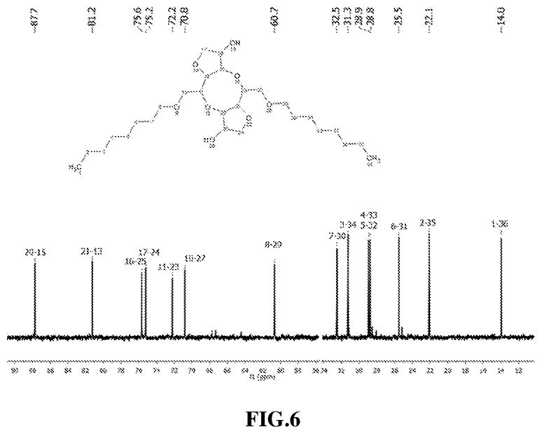

To verify the reactivity of both cycles of the BisCC before initiating polymerization process, we proceed to a model reaction between an alcohol initiator and the D-sorbitol based BisCC (Table 3, Entry 1). After two hours of reaction, the crude media (reaction directly performed in DMSO-d₆) was directly analyzed by ¹H-NMR (FIG. 5), ¹³C-NMR (FIG. 6) and compared to the reactional media ¹H-NMR spectra before reaction (FIG. 4). We observed a full consumption of the five-membered carbonate functions by the absence of the peak at δ (ppm): 5.4 (m, 2H, CH—CH(O)) and δ (ppm): 4.3 (s, 1H, CH₂—OH)) from the 1-octanol. It means that all five-membered cycles were all consumed such as the initiator meaning this molecule has a high potential as cross-linker agent as liberated OH from the reaction are available to react on another BisCC. From Formula 5, this means that three main molecules (1), (2), (3) can be theoretically obtained. Molecule (1) and (2) do not have similar carbons in the cycle meaning that the corresponding carbons peaks are missing on the recorded spectra. Then according to ¹³C-NMR spectra (FIG. 6), the main reaction product is molecule (3). This molecule is bearing two available hydroxyl groups for further reactions like the synthesis of advanced tunable polyurethane or polyester in function of the chosen anionic initiator.

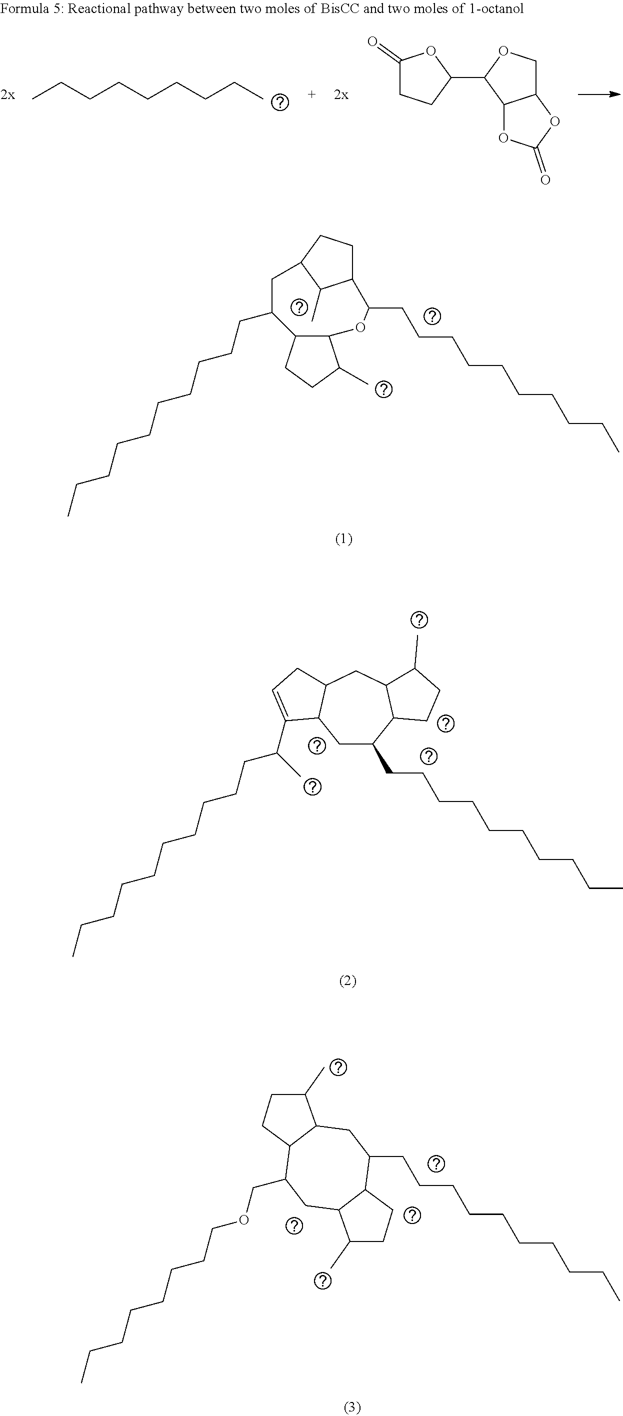

The elaboration of new building blocks through carbonate opening show the potential of the BisCC to be used in polymer synthesis like polyether or polycarbonate by ring opening polymerization (ROP) of the carbonates.

Anionic ROP were firstly conducted with 100, 50, 25, 10 equivalents of five-membered BisCC toward the initiator with 0.7% mol of catalyst (Table 3, Entries 6-9). Brown viscous products were obtained for Entries 6 and 7 (Table 3) and sticky brown material for Entries 8 and 9 (Table 3). SEC analysis of products are indicating two main populations (Table 4) of oligomers with respective degrees of polymerization (DPn) of 5 and 10 based on equation (1). For Entry 9 (Table 4) only one population is detected with a DPn of 5 (Table 3). In some case (Table 4, Entries 6, 7, 9) a small peak was observed with very low molar mass (i.e. 355 g/mol) corresponding to residually initiated BisCC. This similarity of DPn for Entries 6 to 8 (Table 4) indicates that working with 25 equivalents of five-membered BisCC toward the initiator is the best option as higher DPn was not obtained with higher ratio. The main advantage of the reaction is the release of hydroxyl groups each time a cyclic carbonate is open. According to the opening mechanism (FIG. 3) both primary (I-OH) and secondary hydroxyl groups (II-OH) can be obtained. The hydroxyl contents of each oligomer were obtained by quantitative ³¹P-NMR (SI 3) and showed a low content in I-OH, compared to II-OH. The global OH value is comprised between 250 mg KOH/g and 305 mg KOH/g. These OH values and molar masses are promising for the synthesis of advanced polyurethane from these oligomers which could be considered as polyols.

A second set of anionic ROP was conducted with a higher content in catalyst in order to increase the reaction rate (Table 3, Entries 2-4). In each case, the media freeze and brittle brown material was obtained. The conversion of the BisCC was highlighted by ATR-FTIR (SI 2) analysis, the characteristic peak at 1778 cm⁻¹ (C═O, five-membered cyclic carbonates) was absent in each product and a large signal at 3200 cm−1 corresponding to O—H elongation appear. These three products were insoluble in most of the common solvent even after acetylation due to cross-linked materials and in perfect agreement with the reactional mechanism previously establish (Formula 4), the increase of catalyst content increases the reaction rate between the oligomers (Formula 5).

TABLE 4
Properties of main synthetized oligomers

| | | | | | | Global OH | | | | |
|---|---|---|---|---|---|---|---|---|---|---|
| | Mn₁ | Mn₂ | Mn₃ | I-OH content | II-OH content | value | | | Chars | |
| Entry | (g/mol) | (g/mol) | (g/mol) | (mmol/g) | (mmol/g) | (mg KOH/g) | T_{dég 5%} | T_{dég 50%} | (% wt) | DPn |
| 2 | Insoluble | | | | | | 140 | 440 | 0 | — |
| 3 | | | | | | | 98 | 200 | 25 | — |
| 4 | | | | | | | 92 | 271 | 35 | — |
| 5 | 1588 | — | 196 | 0.55 | 4.6 | 288 | 114 | 196 | 0 | 7 |
| 6 | 335 | 1355 | 258 | 0.85 | 4.1 | 276 | 138 | 258 | 0 | 10 |
| 7 | 335 | 1340 | 2315 | 0.68 | 4.2 | 273 | 90 | 228 | 2 | 10 |
| 8 | 1385 | 2500 | — | 0.76 | 4.7 | 305 | 125 | 248 | 8 | 10 |
| 9 | 255 | 1280 | — | 0.85 | 3.6 | 250 | 119 | 244 | 9 | 5 |

### Example 10: Non-Isocyanate Polyurethane Characterization

The synthetized BisCC is also a potential molecule for the synthesis of fully bio-based NIPU. As the BisCC is based on an internal ether cycle and then present high melting point (214° C.), this building block is a source of rigidity for NIPU.

Various diamine from C4 to C36 were used to synthetized NIPU with tunable thermal (i.e., Tg) and chemical properties (i.e. hydroxyl content). 1,4 B and 1,5 MD are two interesting diamines with an equal distance between the two amines groups. However, the 1,5 MD bears a methyl group in position 2 which is really effective to decrease of 36° C. the resulting material Tg (Table 5, Entry 2) compare to NIPU synthetized from 1,4 B (Table 5, Entry 3). On the contrary, the NIPU obtained from P1075 (is a fatty diamine with pendant side chains) presents a low Tg of around −3° C. (Table 5, Entry 1). Beyond the Tg, the OH value of resulting NIPU is dependent of the diamine chain length meaning that NIPU based on P1075 have a low content on hydroxyl groups (3.25 mmol/g) whereas NIPU based on 1,5 MD or 1.4D have a OH value more than two times higher (8.69 mmol/g and 8.84 mmol/g, respectively). Hydroxyl groups are present all along the polymer chain creating functionality on the polymer which can be used to crosslink the NIPU or, as grafting site to introduce novel functions and then properties to the material.

TABLE 5
Thermal and chemical properties of synthetized NIPU

| | | | | | Total OH | | | |
|---|---|---|---|---|---|---|---|---|
| | Diamine | Diamine/BisCC | Tg | T_{deg 50%} | content | Mn | Mw | Dispersity |
| Entry | content | Molar ratio | (° C.) | (° C.) | (mmol/g) | (g/mol) | (g/mol) | Ð |
| 1 | P1075 | 1 | −3 | 342 | 3.25 | 2430 | 5370 | 2.2 |
| 2 | 1,5 MD | 1 | 5 | 213 | 8.69 | 2270 | 5850 | 2.6 |
| 3 | 1,4 B | 1 | 41 | 215 | 8.84 | 1910 | 4480 | 2.4 |
| 4 | P1075/1,5 MD | 0.8/0.2 | −2.4 | 304 | 4.06 | 2770 | 5040 | 1.8 |
| 5 | P1075/1,5 MD | 0.6/0.4 | 2 | 324 | 4.48 | 2140 | 4870 | 2.3 |
| 6 | P1075/1,5 MD | 0.4/0.6 | 20 | 276 | 5.15 | 2400 | 5520 | 2.3 |
| 7 | P1075/1,5 MD | 0.2/0.8 | 37 | 270 | 6.01 | 1500 | 5180 | 3.5 |
| 8 | P1075/1,4 B | 0.8/0.2 | −9 | 318 | 3.90 | 1820 | 3120 | 1.7 |
| 9 | P1075/1,4 B | 0.6/0.4 | −4 | 326 | 4.58 | 1930 | 4360 | 2.3 |
| 10 | P1075/1,4 B | 0.4/0.6 | 12 | 279 | 5.26 | 1840 | 4190 | 2.3 |
| 11 | P1075/1,4 B | 0.2/0.8 | 20 | 257 | 6.26 | 1670 | 5030 | 3 |
| 12 | 1,6 H | 1 | 41 | 224 | 4.83 | 1450 | 2720 | 1.9 |

NIPU's thermal properties recorded by TGA are also dependent on the diamine type. P1075-based NIPU shows higher T_{deg50%} than the 1,5 MD and 1,4 B based NIPU (Table 5, Entry 1-3). This is related to the diamine chain length, P1075 is a 36 carbons fatty acid dimer lowering the global content on urethane function compared to the 4 carbons chain length diamine The urethane function presents a reversibility at lowest temperature than carbon-carbon bonds cleavage. Then NIPU with the highest content in urethane function will be more temperature sensitive and it will start to lose weight early with the molar mass reduction. Even if P1075-based NIPU has a higher thermal stability, there are very soft material due to the P1075 structure based on fatty acid dimer. In order to increase the material hardness, blending of short diamine and P1075 for NIPU synthesis was an effective way to adapt thermal properties. The Tg, T_{deg50%} or chemical properties like the hydroxyl content were tuned to offer a large polymers panel.

In order to adapt the material hardness at ambient temperature, a blend of short diamine and P1075 was an effective way to adapt NIPU's glass temperature from −3° C. to 41° C. Add to the Tg , T_{deg50%} or the hydroxyl content were tuned through these diamines blend to offer a large properties panel for the same range of NIPU.

Materials and Methods

Materials

D-sorbitol was provided by Tereos (MERITOL®, 98%, water content inf. 0.5%, reducing sugar content inf. 0.1%). 1.3.5-triazabicyclo [4.4.0]des-5-ene (TBD), 1.8-diazabicyclo[5.4.0]undec7-ene (DBU), tert-butylimino-tri(pyrrolidino)phosphorarane (purity≥97%, BTPP), dimethyl carbonate (purity≥99%, DMC), diethyl carbonate (purity≥99%, DEC) ethylene carbonate (purity 99+%, EC) and 1-octanol (purity≥99%)) were obtained from Sigma Aldrich. Sodium hydroxide (NaOH) was obtained from Carlo Erba Reagents. Hydroxide potassium (KOH) and potassium carbonate (K2CO3), 1.5-diamino-2-methylpentane (99%) were obtained from VWR Chemical. Titanium (IV) isopropoxide (TTIP), titanium (IV) butoxide (TNBT) and stannous octoate were obtained from Acros Organics. 1.4 diaminobutane (98+%) was obtained from Alfa Aesar. Hexamethylene diamine (1.6 H, 98%) was provided by BASF. Priamine 1075 amine value 3.64 mmol/g) was kindly provided by Croda. All reagents were used without further purification.

Methods

¹H- and ¹³C-NMR spectra were performed with a Bruker 400 MHz. Deuterated dimethyl sulfoxide (DMSO-d₆) was used as solvent to prepare sample solutions with concentrations of 8-10 and 20-30 mg/mL for ¹H-NMR and ¹³C-NMR, respectively. The number of scans was set to 64 for ¹H-NMR and at least 2048 for ¹³C-NMR. The calibration of ¹H- and ¹³C-NMR spectra were performed using the DMSO peak at 2.50 and 39.52 ppm, respectively. Water molecules present in DMSO-d₆ induced a supplementary peak in ¹H-NMR spectra at 3.33 ppm.

³¹P-NMR analysis were performed with a Bruker 400 MHz spectrophotometer after phosphitylation of the sample with 2-chloro-4,4,5,5-tetramethyl-1,3,2-dioxaphospholane according to standard protocols ^{17,18}, the number of scans was set to 128 at 25° C. Peak analysis and quantitative analysis were performed according to previous reports¹⁹.

Elementary analysis was performed on a ThermoFisher Scientific “Flash 2000” device (absolute precision of 0.3%) with 1 mg sample burn up to 950° C.

Electrospray ionization mass (MS) experiments were performed on a Bruker Daltonics microTOF spectrometer (Bruker Daltonik GmgH, Bremen, Germany) equipped with an orthogonal electrospray (ESI) interface. Calibration was performed using a solution of 10 mM sodium formiate. Sample solutions were introduced into the spectrometer source with a syringe pump (Harvard type 55 1111: Harvard Apparatus Inc., South Natick, Mass., USA) with a flow rate of 5 μL·min⁻¹.

Acetylations were performed in a pyridine/acetic anhydride mixture (1:1 v/v) at room temperature for 24 h to increase sample solubility for analysis as previously reported²⁰.

Differential scanning calorimetry (DSC) was performed on a TA Instrument Q200 under nitrogen flux (50 mL/min). Samples of 1-3 mg were sealed in hermetic aluminum pans and analyzed using cyclic procedure involving a heating ramp at 10° C./min, a cooling ramp at 5° C./min, then a second heating at 10° C./min. Between each ramp, the temperature was hold 2 min for stabilization.

Thermogravimetric analyses (TGA) were performed using a TA Instrument Hi-Res TGA Q5000 under helium (flow rate 25 mL/min) and/or reconstituted air (flow rate 25 mL/min). Samples of 1-3 mg were heated from room temperature to 600° C. (10° C./min). The main characteristic degradation temperatures are those at the maximum of the weight loss derivative curve (DTG) (T_{deg}).

Infrared spectroscopy was performed with a Fourier transformed infrared spectrometer Nicolet 380 used in reflection mode equipped with an ATR diamond module (FTIR-ATR). An atmospheric background was collected before each sample analysis (32 scans, resolution 4 cm⁻¹).

Size exclusion chromatography measurements were performed in tetrahydrofuran (THF, HPLC grade) in Waters Acquity APC system equipped with a 1.7 μm, 45 Å, 150 mm APC XT column, 2.5 μm, 200 Å 150 mm APC XT column and a 2.5 μm, 450 Å, 150 mm APC XT column, a Acquity RI refractive index detector and a Acquity TUV diode array (UV) detector. The instrument was calibrated with linear polystyrene standards from 162 to 1,650,000 g/mol and reported molar masses are the molar masses at the peak. Sample presenting low solubility in THF were prior acetylated to performed analysis. In the case of small molar mass (i.e., oligomers) the degree of polymerization (DPn) is evaluated according to equation (1).

$$D P n = \frac{( M n - M {n}_{ini} )}{M {n}_{0}} \qquad ( 1 )$$

Where Mn is the obtain number-average molar mass, Mnᵢₙᵢ the molar mass of the initiator and Mn₀ the molar mass of the monomer.

### CONCLUSION

A yield of 50% of D-sorbitol based bis-cyclocarbonate was successfully obtained using fully bio-based reactant in bulk condition under 80° C. A fruitful alliance has been found between D-sorbitol, DMC and TBD thanks to the TBD solubility in DMC at 75° C. The optimum yield was obtained by setting up a reactional device with continuous feeding of DMC to limit the azeotrope formation between reactant (DMC) and reactional subsidiary product (MeOH). Then the versatility of the carbonate chemistry permitted us to obtain two family of products:
a promising and tunable molecule for advanced polymer synthesis and polyethers/polycarbonates oligomers. Polyethers/polycarbonates were obtained by anionic ROP of the synthesized D-sorbitol based five-membered BisCC;
the synthetized BisCC is also a promising building block for NIPU synthesis.

### BIBLIOGRAPHY

1 J. E. Holladay, J. J. Bozell, J. F. White and D. Johnson, DOE Rep. PNNL, 2007, 16983.
2 J. J. Bozell and G. R. Petersen, Green Chem., 2010, 12, 539.
3 A. Anand, R. D. Kulkarni and V. V. Gite, Prog. Org. Coat., 2012, 74, 764-767.
4 L. Maisonneuve, O. Lamarzelle, E. Rix, E. Grau and H. Cramail, Chem. Rev., 2015, 115, 12407-12439.
5 C. Carré, H. Zoccheddu, S. Delalande, P. Pichon and L. Avérous, Eur. Polym. J., 2016, 84, 759-769.
6 A. Cornille, G. Michaud, F. Simon, S. Fouquay, R. Auvergne, B. Boutevin and S. Caillol, Eur. Polym. J., 2016, 84, 404-420.
7 M. Tryznowski, A. Świderska, Z. Żołek-Tryznowska, T. Gołofit and P. G. Parzuchowski, Polymer, 2015, 80, 228-236.
8 M. Wu, J. Guo and H. Jing, Catal. Commun., 2008, 9, 120-125.
9 O. Haba, H. Tomizuka and T. Endo, Macromolecules, 2005, 38, 3562-3563.
10 K. Tezuka, K. Koda, H. Katagiri and O. Haba, Polym. Bull., 2015, 72, 615-626.
11 P. Tundo and M. Selva, Acc. Chem. Res., 2002, 35, 706-716.
12 U. Romano, F. Rivetti and N. Di Muzio, Process for producing dimethylcarbonate, Google Patents, 1982.
13 U. Romano and F. Rivetti, Process for preparing di-alkyl carbonates, Google Patents, 1993.
14 I. Garcia-Herrero, R. M. Cuéllar-Franca, V. M. Enríquez-Gutiérrez, M. Alvarez-Guerra, A. Irabien and A. Azapagic, ACS Sustain. Chem. Eng., 2016, 4, 2088-2097.
15 L. Hough, J. E. Priddle and R. S. Theobald, J. Chem. Soc. Resumed, 1962, 1934-1938.
17. P. Dais and A. Spyros, Magn. Reson. Chem., 2007, 45, 367-377.
18. D. S. Argyropoulos, Res. Chem. Intermed., 1995, 21, 373-395.
19. A. Granata and D. S. Argyropoulos, J. Agric. Food Chem., 1995, 43, 1538-1544.
20. A. Arbenz and L. Avérous, Ind. Crops Prod., 2015, 67, 295-304.
21. M. M. Mazurek-Budzyńska, G. Rokicki, M. Drzewicz, P. A. Guńka and J. Zachara, Eur. Polym. J., 2016, 84, 799-811.
22. N. Jacquel, F. Freyermouth, F. Fenouillot, A. Rousseau, J. P. Pascault, P. Fuertes and R. Saint-Loup, J. Polym. Sci. Part Polym. Chem., 2011, 49, 5301-5312.
23. V. Tserki, P. Matzinos, E. Pavlidou, D. Vachliotis and C. Panayiotou, Polym. Degrad. Stab., 2006, 91, 367-376.
24. R. Schwesinger, J. Willaredt, H. Schlemper, M. Keller, D. Schmitt and H. Fritz, Chem. Ber., 1994, 127, 2435-2454.
25. J. H. Clements, Ind. Eng. Chem. Res., 2003, 42, 663-674.

1-15. (canceled)

16. A process for producing a five-membered polycycloaliphatic carbonate comprising a step of reacting a suspension of a powdered sugar alcohol within a carbon dioxide source and a catalyst compound which is soluble in said carbon dioxide source at the reactional temperature.

17. The process according to claim 16, wherein the powdered sugar alcohol selected among erythritol, arabitol, xylitol, ribitol, D-sorbitol, dulcitol, D-mannitol, volemitol, maltitol, isomalt, lactitol and a mix thereof, typically said powdered sugar alcohol is a crystalline sugar alcohol.

18. The process according to claim 16, wherein said carbon dioxide source is a linear dialkyl carbonate or a cyclic carbonate with the general formula R1-O—CO—O—R2 wherein said alkyl groups R1 and R2 are each independently selected from the group consisting of C1-C4 alkyl group, benzyl group or a phenyl group, optionally wherein R1 and R2 is covalently linked to form a cyclic carbonate.

19. The process according to claim 18, wherein said carbon dioxide source is selected in the list of dimethyl carbonate, diethyl carbonate, diphenyl carbonate, dibenzyl carbonate, propylene carbonate, ethylene carbonate, 1,2-butylene carbonate, glycerol carbonate, 4,5-dimethyl-1,3-dioxolan-2-one and the mix thereof.

20. The process according to claim 16, wherein said five-membered polycycloaliphatic carbonate is a five-membered biscycloaliphatic carbonate or a tricycloaliphatic carbonate.

21. The process according to claim 16, wherein the reactional temperature is between 20° C.-250° C.

22. The process according to claim 16, wherein the reactional temperature is between 30° C.-200° C.

23. The process according to claim 16, wherein the catalyst is a basic catalyst, preferably an organo-basic-catalyst.

24. The process according to claim 16, wherein the catalyst is a an organo-basic-catalyst.

25. The process according to claim 16, wherein the carbon dioxide source is dimethyl carbonate, the powdered sugar alcohol is a powdered D-sorbitol and the five-membered polycycloaliphatic carbonate is the bis(cyclo-carbonate) (BisCC).

26. The process according to claim 16, wherein the ratio of carbon dioxide source/sugar alcohol ranges from 1 to 18.

27. The process according to claim 16, wherein the ratio of carbon dioxide source/sugar alcohol ranges from 2 to 9.

28. The process according to claim 16, wherein the ratio of carbon dioxide source/sugar alcohol ranges from 3 to 8.

29. The process according to claim 16, wherein the carbon dioxide source is added by continuous feeding.

30. The process according to claim 16, wherein said process further comprises the additional steps of:
precipitating the five-membered polycycloaliphatic carbonate in an aqueous composition;
optionally, washing the precipitated five-membered polycycloaliphatic carbonate obtained; and
recovering the five-membered polycycloaliphatic carbonate by filtrating and/or drying the precipitate obtained.

31. A process for producing non-isocyanate polyurethane and/or a polycarbonate and/or polyethers comprising the steps of:
obtaining a five-membered polycycloaliphatic carbonate following the process steps according to claim 1; and
mixing said five-membered polycycloaliphatic carbonate with a compound comprising at least one amine function or one alcohol function.

32. The process according to claim 31, wherein the compound comprising at least one alcohol function is selected from the group consisting of methan-1-ol, ethan-1-ol, propan-1-ol, butan-1-ol, hexan-1-ol, octan-1-ol, decan-1-ol, dodecan-1-ol, 1,3-propandiol, 1,4-butanediol, 1,6-hexanediol, 1,8-octanediol, 1,10-decanediol, 1,12-dodecanediol and the mix thereof.

33. The process according to claim 31, wherein the compound comprising at least one amine function, is selected from the group consisting of a dimer-based diamine having 36 carbon atoms , a polyetherdiamine compound having a molar mass between 200 and 5000 g/mol, a polyethertriamine having a molar mass between 400 and 5000 g/mol , 4,9-dioxa-1,12-dodecanediamine, 1,12-diaminododecane, 1,10-diaminodecane, 1.8-diaminooctane, 1,6-diaminohexane, 1,5-diaminopentane, 1,5-diamino-2-methylpentane, 1,4-diaminobutane, hexamethylene diamine and the mix thereof.
